(19) United States
(12) Patent Application Publication
(10) Pub. No.: US 2014/0303246 A1
(43) Pub. Date: Oct. 9, 2014

(54) USE OF ADAMANTANE DERIVATIVES FOR THE TREATMENT OF ACTINIC KERATOSIS

(71) Applicants: Kevin Kiehm, Telgte (DE); Rainer Pooth, Bad Soden/Taunus (DE); Harry Frank Abts, Oberursel (DE)

(72) Inventors: Kevin Kiehm, Telgte (DE); Rainer Pooth, Bad Soden/Taunus (DE); Harry Frank Abts, Oberursel (DE)

(73) Assignee: MERZ Pharma GmbH & Co. KGaA, Frankfurt am MAIN (DE)

(21) Appl. No.: 14/343,162

(22) PCT Filed: Sep. 7, 2012
(86) PCT No.: PCT/EP2012/067535

§ 371 Date: Apr. 29, 2014

Related U.S. Application Data
(60) Provisional application No. 61/532,160, filed on Sep. 8, 2011.

(30) Foreign Application Priority Data
Sep. 8, 2011 (EP) 11180630.3

(51) Int. Cl. A61K 31/13; A61K 31/21

(52) U.S. Cl. CPC A61K 31/13; A61K 31/21
USPC 514/507; 514/662; 514/659

(57) ABSTRACT

Use of an adamantaneamine derivative for the treatment of actinic keratosis.

The present invention relates to the use of adamantane derivatives for the treatment of actinic keratosis.

In general, actinic keratosis (also called “solar keratosis” and “senile keratosis”, AK) is a premalignant condition of thick, scaly, or crusty patches of skin consisting of dysplastic keratinocytic lesions. AK is one of the most common conditions treated by dermatologists. It is more common in fair-skinned people. And most important it is associated with those who are frequently exposed to the sun, as it is usually accompanied by solar damage. It is generally accepted that these lesions can progress to squamous cell carcinoma (SSC). Concerning the rate of this transformation there is a controversy in the literature. Annual rates of transformation are ranging from 0.1% -20%. Nevertheless there is no doubt that these pre-cancerous lesions should be treated. In addition lesions are in general treated also for cosmetic purposes and to provide relief from symptoms, such as tenderness or itch.

When skin is exposed to the sun constantly, thick, scaly, or crusty bumps may appear. The scaly or crusty part of the bump is dry and rough. The growths start out as flat scaly areas, and later grow into a tough, wart-like area.

In addition to chronic UV-exposure also infections with HPV has been implicated in the aetiology of AK.

An actinic keratosis site commonly ranges between 2 and 6 millimeters in size, and can be dark or light, tan, pink, red, a combination of all these, or have the same pigment as the surrounding skin. It typically appears on any sun-exposed area, such as the face, ears, neck, scalp, chest, backs of hands, forearms, or lips.

An efficient treatment that can be used to treat larger areas of affected skin and eliminate obvious AK lesions as well as clinically non visible pre-lesions would be beneficial for the patient.

Therefore, it is an object of the present invention to provide alternative treatments for actinic keratosis.

This object is solved by claim 1 of the present invention. Accordingly, the use of adamantane derivatives of the structure R₁-[CR₂R₃]ₙ—NR₄R₅ and/or pharmaceutically acceptable salts thereof for the treatment of actinic keratosis is provided, whereby

R₁ is unsubstituted or alkyl, long-chain alkyl, alkoxy, long-chain alkoxy, cycloalkyl, halogenalkyl, aryl, arylene, halogenaryl, and/or halogen mono, di or polysubstituted adamantane, whereby each substitution is independent from the other in case of more than one substituent

R₂ and R₃ are independently from each other hydrogen, alkyl, long-chain alkyl, alkoxy, long-chain alkoxy, cycloalkyl, halogenalkyl, aryl, arylene or halogenaryl, whereby in case n>1 every R along the carbon chain may differ from each other

n is an integer from 0 to 6

R₄ is hydrogen, alkyl, long-chain alkyl, alkoxy, long-chain alkoxy, cycloalkyl, halogenalkyl, aryl, arylene or halogenaryl, carbony or —CO—O—R_{4′}, with R_{4′} being alkyl and/or long-chain alkyl,

R₅ is hydrogen, alkyl, long-chain alkyl, alkoxy, long-chain alkoxy, cycloalkyl, halogenalkyl, aryl, arylene or halogenaryl, —O—[CR₆R₇]ₘ—NR₈R₉, or —O—[CR₆R₇]ₘ—SR₈, whereby m is an integer from 1 to 5, R₆ and R₇ are independently from each other hydrogen, alkyl, cycloalkyl or halogenalkyl, whereby in case m>1 every R along the carbon chain may differ from each other; R₈ and R₉ are independently from each other hydrogen, alkyl, cycloalkyl or halogenalkyl,

whereby for any R at suitable residues one or more CH₂-groups may independently from each other substituted by —O—, —S—, —NH—, —NR^{o}—, —SiR^{o}R^{oo}—, —CO—, —COO—, —OCO—, —OCO—O—, —SO₂—, —S—CO—, —CO—S—, —CY¹═CY² oder —C≡C— in that way that O and/or S atoms are not directly bound to each other; terminal CH₃.groups are understood as CH₂—H groups.

Surprisingly it has been found that compounds of this structure can be used for the treatment of actinic keratosis. Without being bound to any theory the inventors believe that this at least partly due to a multiple synergistic effect since many compounds of these structure have found to have at least two, mostly all three of the following effects:
keratinolytic activity
immunomodulation and thus likely stimulation of TLR7-receptor
antiviral activity

Generic group definition: Throughout the description and claims generic groups have been used, for example alkyl, alkoxy, aryl. Unless otherwise specified the following are preferred groups that may be applied to generic groups found within compounds disclosed herein:

alkyl: linear and branched C1-C8-alkyl,

long-chain alkyl: linear and branched C5-C20 alkyl

alkenyl: C2-C6-alkenyl,

cycloalkyl: C3-C8-cycloalkyl,

alkoxy: C1-C6-alkoxy,

long-chain alkoxy: linear and branched C5-C20 alkoxy

alkylene: selected from the group consisting of:

methylene; 1,1-ethylene; 1,2-ethylene; 1,1-propylidene; 1,2-propylene; 1,3- propylene; 2,2-propylidene; butan-2-ol-1,4-diyl; propan-2-ol-1,3-diyl; 1, 4-butylene; cyclohexane-1,1-diyl; cyclohexan-1,2-diyl; cyclohexan-1,3-diyl; cyclohexan-1,4-diyl; cyclopentane-1,1-diyl; cyclopentan-1,2-diyl; and cyclopentan-1,3-diyl,

aryl: selected from homoaromatic compounds having a molecular weight under 300,

halogen: selected from the group consisting of: F; Cl; Br and I,

halogenalkyl: selected from the group consisting of mono, di, tri-, poly and perhalogenated linear and branched C1-C8-alkyl carbonyl: the group —C(O)R, wherein R is selected from: hydrogen; C₁-C₆-alkyl; phenyl; C₁-C₆-alkyl-C₆H₅ and amine (to give amide) selected from the group: —NR′₂, wherein each R′ is independently selected from: hydrogen; C₁-C₆-alkyl; C₁-C₆-alkyl-C₆H₅; and phenyl, wherein when both R′ are C₁-C₆-alkyl both R′ together may form an —NC₃ to an —NC₅ heterocyclic ring with any remaining alkyl chain forming an alkyl substituent to the heterocyclic ring,

Unless otherwise specified the following are more preferred group restrictions that may be applied to groups found within compounds disclosed herein:

alkyl: linear and branched C1-C6-alkyl, more preferred methyl, ethyl, propyl, isopropyl, buyl, isobutyl

long-chain alkyl: linear and branched C5-C10 alkyl, preferably linear C6-C8 alkyl

alkenyl: C3-C6-alkenyl,

cycloalkyl: C6-C8-cycloalkyl,

alkoxy: C1-C4-alkoxy,

long-chain alkoxy: linear and branched C5-C10 alkoxy, preferably linear C6-C8 alkoxy

alkylene: selected from the group consisting of: methylene; 1,2-ethylene; 1,3-propylene; butan-2-ol-1,4-diyl; 1,4-butylene; cyclohexane-1,1-diyl; cyclohexan-1,2-diyl; cyclohexan-1,4-diyl; cyclopentane-1,1-diyl; and cyclopentan-1,2-diyl,

aryl: selected from group consisting of: phenyl; biphenyl; naphthalenyl; anthracenyl; and phenanthrenyl,

halogen: selected from the group consisting of: F and Cl,

carbonyl: the group: —C(O)R, wherein R is selected from: hydrogen; C1-C6-alkyl; benzyl and amine selected from the group: —NR′2, wherein each R′ is independently selected from: hydrogen; C1-C6-alkyl; and benzyl,

The term “adamantane” relates to the chemical moeity with the following structure:

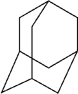

It should be noted that the —[CR₂R₃]ₙ—NR₄R₅ unit may be bound either to one of the four tertiary or to one of the six secondary carbon atoms of the adamantane; however, it is preferred that the unit is bound to a tertiary carbon atom.

Synthesis methods and instructions of the adamantane derivatives of the present invention are well-known in the art. In this regard it should only be mentioned that adamantane can be substituted at the tertiary position using synthesis pathways involving carbocations or similar species. Adamantane can e.g. brominated easily at the tertiary 1-position using Br₂ and a catalyst such as FeBr₃ or AlBr₃/AlCl₃. Also carboxylation using formic acid under acidic condition is possible.

Substitution of the secondary positions is e.g. possible by first oxidizing the adamantane to adamantane-2-one (e.g. with concentrated sulfuric acid) and then e.g. reductively aminating the ketone (or performing other suitable reactions).

In case that n=1 or higher, often 1-adamantanoylchloride is used as starting compound (et the synthesis of rimantadine as described in U.S. Pat. No. 4,551,552 and cited prior art therein).

The term “pharmaceutically acceptable salts thereof” especially means and/or includes that in case that the adamantane derivative according to the preferred invention forms a quarternary ammonium salt (i.e. in case that R₄ and R₅ are both either H or alkyl although this is not limiting) that also a pharmaceutically acceptable salt may be used instead of the free base. Pharmaceutically acceptable salts may include—although not limited to—chlorides, sulfates, citrates, tatrates and/or salts of the following acids: acetic, aceturic, adipic, carbonic, fumaric, galactaric, glucaric, gluconic, glucoronic, glutamic, glutaric, glycolic, hippuric, hydrochloric, hydobromis, lactic, lactobionic, lauric, maleic, malic, palmitic, phosphoric. Pyruvic, succinic, sebacic, sulfuric, stearic, tartaric or thiocyanic acids.

According to an embodiment of the invention it is preferred that n is 0 or 1 with R₂=H and R₃=alkyl in case that n=1.

According to an embodiment of the invention it is preferred that R₄=carbonyl and R₅=H, alkoxyl or —O—[CR₆R₇]ₘ—NR₈R₉ (with R₆ to R₉ as explained above). According to an embodiment of the invention, the adamantane derivative is chosen out of the group comprising amantadine, tromantadine, rimantadine, memantine or mixtures thereof.

Amantadine is adamantan-1-amine and has the following structure:

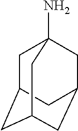 [SMILES: NC12CC3CC(CC(C3)C1)C2]

Tromantadine is N-1-adamantyl-N-[2-(dimethylamino)ethoxy]acetamide and has the following structure:

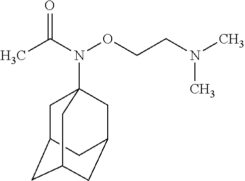 [SMILES: CC(=O)N(OCCN(C)C)C12CC3CC(CC(C3)C1)C2]

Rimantadine is 1-(1-adamantyl)ethanamine and has the following structure:

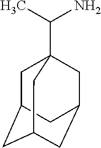

It is a chiral compound and can be used either as the racemate and/or one of the enantiomers.

Memantine is 3,5-dimethyladamantan-1-amine and has the following structure:

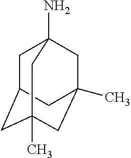

Application of a formulation of the present invention carried out by any form of injection or topical application, in particular by subcutaneous injection.

The adamantane amine derivative can be applied topically. Examples for a topic application of a formulation comprising a adamantane amine derivative include a cream, a patch, a salve, a gel, a powder, a dressing, ointment, iontophoresis or transdermal system.

Preferably the concentration of the adamantane derivative is from about 0.1% to about 20% (wt/wt), preferred 1% to 10%.

Alternatively, the adamantane amine derivative can be applied by subcutaneous injection. Examples for subcutaneous injection include aqueous solutions, suspensions, oily solutions, emulsions, microemulsions, liposomes, microspheres, nanoparticles and implants. The advantage of subcutaneous injections is the rapid onset of action and that the cytolytic effect is restricted to the targeted tissue. Furthermore, the systemic availability of compounds over time is reduced, since drug absorption from subcutaneous tissue is slow.

Preferably, each injection unit of the formulation has a distinct dose of the adamantane amine derivative according to the invention. This dose can reach from about 50 μMol to about 50 mMol, preferred from about 100 μMol to about 10 mMol, per volume of one injection shot. One injection shot is sized from about 0.15 ml of the formulation to about 2.0 ml, more preferable between 0.5 and 1 ml.

The present invention furthermore relates to a formulation comprising at least one adamantane derivative of the structure R₁-[CR₂R₃]ₙ—NR₄R₅ and/or a pharmaceutically acceptable salt thereof for the treatment of actinic keratosis.

The present invention furthermore relates to process, comprising: administering adamantane derivative of the structure R₁-[CR₂R₃]ₙ—NR₄R₅ and/or a pharmaceutically acceptable salt thereof to a human in an amount effective for treating actinic keratosis

Additional details, characteristics and advantages of the object of the invention are disclosed in the subclaims and the following description of the respective figures and examples.

In an additional embodiment the claimed adamantane derivate is combined with a keratolytic agent in order to support the therapeutic effect. The keratolytic agent could simply be a chemical peel like compound like salicylic-acid ore could have a more efficient keratino-cytolic activity like retinoids.

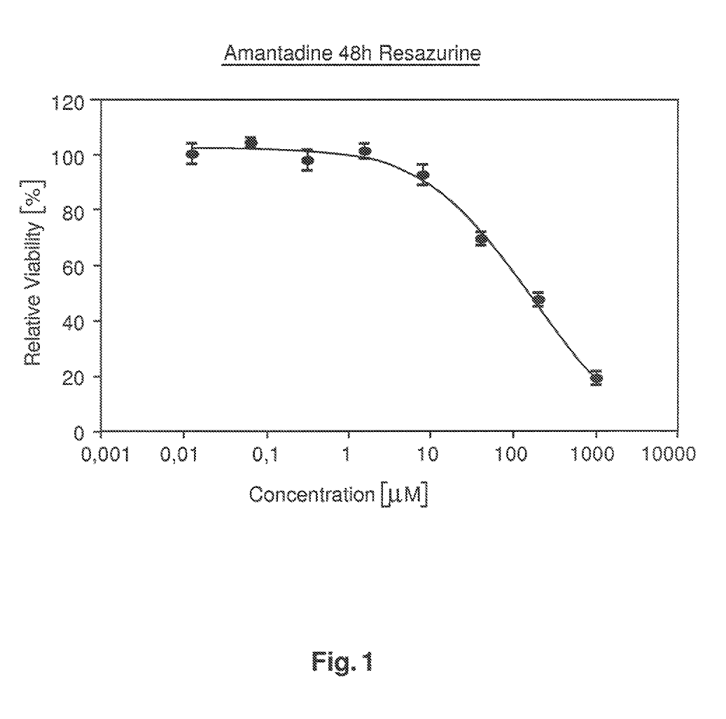

FIG. 1 is a diagram showing the relative viability vs. the concentration for the compound according to Example 1 of the present invention on NHEK cells

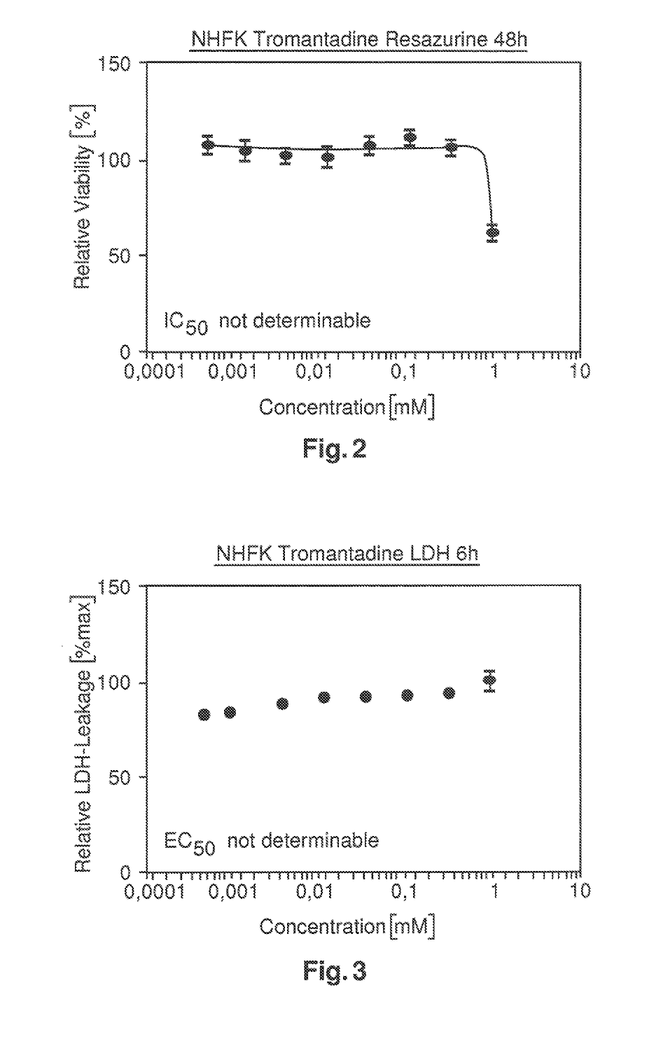

FIG. 2 is a diagram showing the relative viability vs. the concentration for the compound according to Example 2 of the present invention on NHEK cells

FIG. 3 is a diagramm showing the effect on viability based on metabolic activity; Membrane damage (LDH-leakage) after 6 hrs for the compound according to Example 2

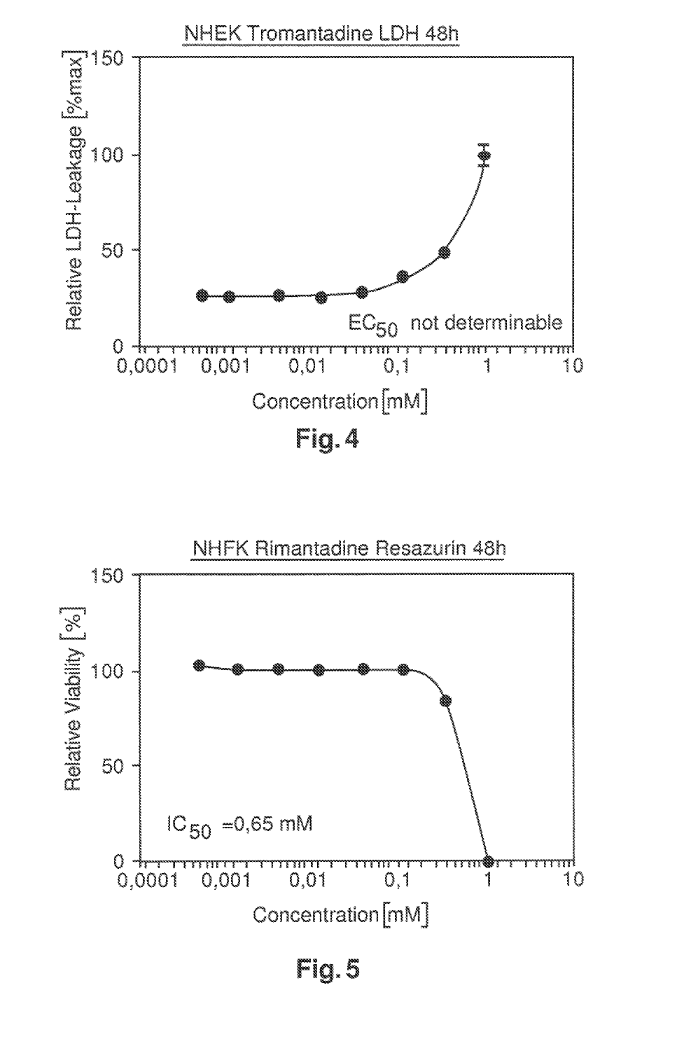

FIG. 4 is a diagramm showing the effect on viability based on metabolic activity; Membrane damage (LDH-leakage) after 48 hrs for the compound according to Example 2

FIG. 5 is a diagram showing the relative viability vs. the concentration for the compound according to Example 3 of the present invention on NHEK cells

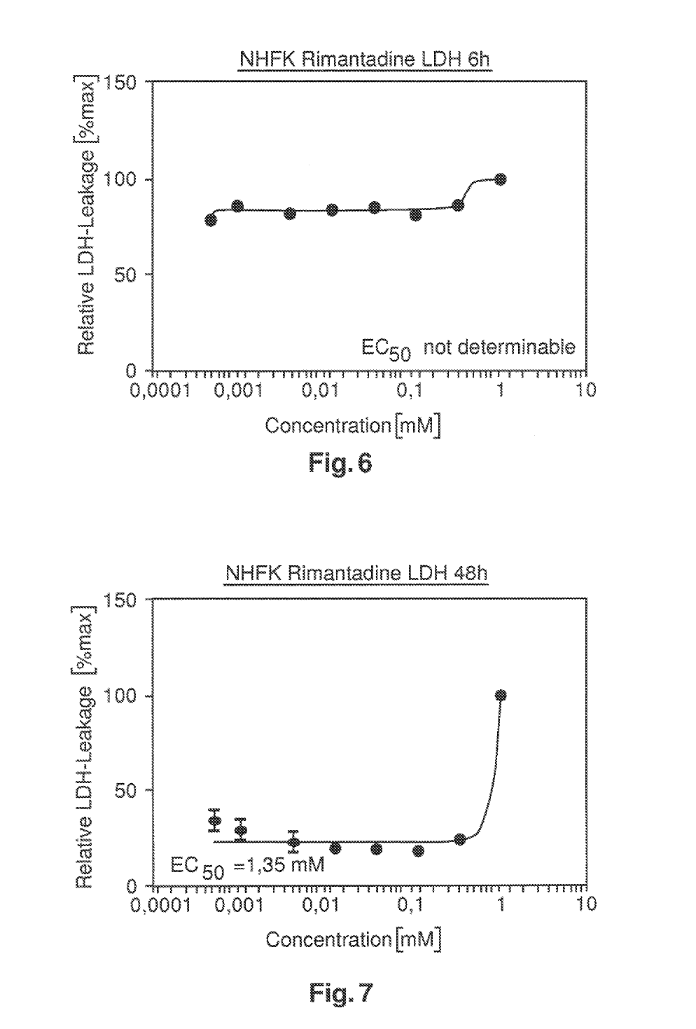

FIG. 6 is a diagramm showing the effect on viability based on metabolic activity; Membrane damage (LDH-leakage) after 6 hrs for the compound according to Example 3

FIG. 7 is a diagramm showing the effect on viability based on metabolic activity; Membrane damage (LDH-leakage) after 48 hrs for the compound according to Example 3

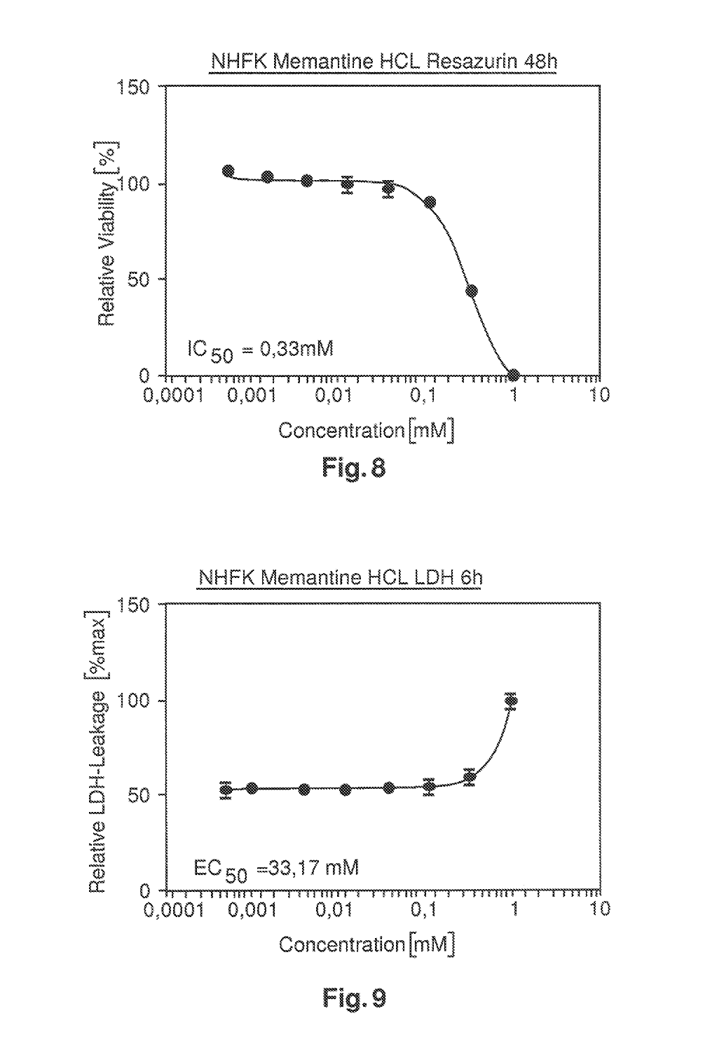

FIG. 8 is a diagram showing the relative viability vs. the concentration for the compound according to Example 4 of the present invention on NHEK cells

FIG. 9 is a diagramm showing the effect on viability based on metabolic activity; Membrane damage (LDH-leakage) after 6 hrs for the compound according to Example 4

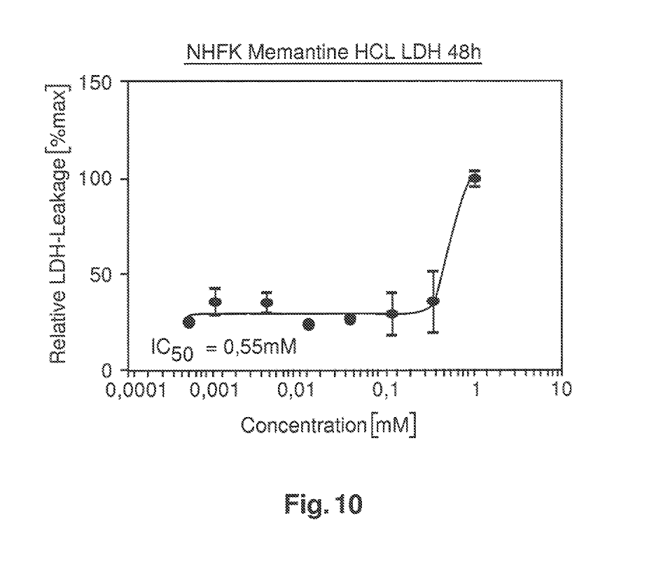

FIG. 10 is a diagramm showing the effect on viability based on metabolic activity; Membrane damage (LDH-leakage) after 48 hrs for the compound according to Example 4

### EXAMPLE 1

Example 1 refers to Adamantadine, whose structure is given above.

By using primary human epidermal keratinocytes (NHEK, FIG. 1) the cytolytic potential of several drug compound was investigated.

The viability of the cells was monitored by their metabolic activity using the Resazurin—assay. Eight different concentrations (up to 1 mM) of amantadine were investigated. Viability of the cells was plotted against the different concentration of test compound.

From the resulting sigmoid-curve the IC50 value for the cytolytic effect was determined and given in the respective figure. FIG. 1 depicts the relative viability vs. the concentration on NHEK cells using metabolic activity as read-out. The obtained data show a clear negative-impact of suggested drug compound on the viability of the cells.

### EXAMPLES 2 to 4

Example 2 refers to Tromantadine, which structure is given above

Example 3 refers to Rimantadine, which structure is given above

Example 4 refers to Memantine, which structure is given above.

For each of these compounds, the same experiments were performed in an analogous fashion to the compound of Example 1 except that the test items were prepared in separate plates starting with the double concentrated maximal test concentration followed by a 1:3 serial dilution and then added to the cells. The test items were tested at 6 replicates/concentration. In addition to metabolic activity also the release of cytoplasmatic-lactate dehydrogenase (LDH) was used to monitor cell death. Early release (after 6 hrs) indicate membrane damage a primary mode of action

Results are given in FIGS. 2 to 4 (Example 2), 5 to 7 (Example 3) and 8 to 10 (Example 4).

Tromantadine (Example 2) shows a cytotoxic effect at high concentrations. From 100 μM on LDH is released from cells after 48 h. The EC50 value for LDH-release after 48 h was determined to be 335 μM.

Rimantadine (Example 3) shows an even more pronounced cytotoxic effect with an IC₅₀ of 660 μM based on metabolic activity as read-out.

Memantine (Example 4) shows also a clear cytotoxic activity on NHEK with an IC50 of 330 μM based on metabolic activity. In contrast to Tromantadine and Rimantadine the LDH-release at 6 hrs indicate an early membrane damage by Memantine.

The obtained data show also for these compounds a clear and potent effect of suggested drug compound on the viability of the cells.

The particular combinations of elements and features in the above detailed embodiments are exemplary only; the interchanging and substitution of these teachings with other teachings in this and the patents/applications incorporated by reference are also expressly contemplated. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. The invention's scope is defined in the following claims and the equivalents thereto. Furthermore, reference signs used in the description and claims do not limit the scope of the invention as claimed.

1-6. (canceled)

7. A method of treating actinic keratosis in a subject in neat thereof comprising administration of an effective amount of an adamantane derivative or a pharmaceutically acceptable salt thereof of the following structure
R₁—[CR₂R₃]ₙ—NR₄R₅
wherein
R₁ is unsubstituted or alkyl, long-chain alkyl, alkoxy, long-chain alkoxy, cycloalkyl, halogenalkyl, aryl, arylene, halogeriaryl, and/or halogen mono, di or polysubstituted adamantane, wherein each substitution is independent from the other in case of more than one substituent.
R₂ and R₃ are independently from each other hydrogen, alkyl, long-chain alkyl, alkoxy, long-chain alkoxy, cycloalkyl, halogenalkyl, aryl, arylene or halogenaryl, wherein in case n>1 every R along the carbon chain may differ from each other
n is an integer from 0 to 6
R₄ is hydrogen, alkyl, long-chain alkyl, alkoxy, long-chain alkoxy, cycloalkyl, halogenalkyl, aryl, arylene or halogenaryl, carbonyl or —CO—O—R_{4′}, with R_{4′} being alkyl or long-chain alkyl,
R₅ is hydrogen, alkyl, long-chain alkyl, alkoxy, long-chain alkoxy, cyclcalkyl, halogenalkyl, aryl, arylene or halogenaryl, —O—[CR₆R₇]ₘ—NR₈R₉, or —O—[CR₆R₇]ₘ—SR₈, wherein m is an integer from 1 to 5, R₆ and R₇ are independently from each other hydrogen, alkyl, cycloalkyl or halogenalkyl, wherein in case m>1 every R along the carbon chain may differ from each other; R₈ and R₉ are independently from each other hydrogen, alkyl, cycloalkyl or halogenalkyl,
wherein for any R at suitable residues one or more CH₂-groups may independently from each other substituted by —O—, —S—, —NH—, —NR^{o}—, —SiR^{o}R^{oo}—, —CO—, —COO—, —OCO—, —OCO—O—, —SO₂—, —S—CO—, —CO—S—, —CY¹═CY² or —C≡C—, wherein O and S atoms are not directly bound to each other; and terminal CH₃-groups are understood as CH₂—H groups.

8. The method according to claim 7, wherein n is 0 or 1.

9. The method according to claim 7, wherein the adamantane derivative is applied topically or in form of an injection.

10. The method according to claim 7, wherein the the dose of adamantane amine derivative per one injection is between 50 μMol and 50 mMol.

11. The method according to claim 7, wherein the R₄ is carbonyl and R₅ is H, alkoxyl or —O—[CR₆R₇]ₘ—NR₈R₉.

12. The method according to claim 7, wherein the adamantane derivative is selected from the group consisting of amantadine, tromantadine, rimantadine, memantine and mixtures thereof.
